(19) United States
(12) Patent Application Publication
(10) Pub. No.: US 2025/0064604 A1
(43) Pub. Date: Feb. 27, 2025

(54) PROSTHETIC DEVICE

(71) Applicant: Aether Biomedical sp. z o.o., Poznan (PL)

(72) Inventor: Kamil FABISZAK, Poznan (PL)

(73) Assignee: Aether Biomedical sp. z o.o., Poznan (PL)

(21) Appl. No.: 18/847,794

(22) PCT Filed: Apr. 18, 2023
(86) PCT No.: PCT/IB2023/053963

§ 371 Date: Sep. 17, 2024

(30) Foreign Application Priority Data
Apr. 20, 2022 (PL) P.440584

(51) Int. Cl. A61F 2/58; A61F 2/50; A61F 2/70

(52) U.S. Cl. CPC A61F 2/586; A61F 2/70; A61F 2002/5083; A61F 2002/5089; A61F 2002/587

(57) ABSTRACT

Disclosed is a prosthetic device. The prosthetic device includes a unitary main body having a plurality of slots, wherein each of the plurality of slots includes an electrical interface; and a plurality of digits configured to be detachably connected to the main body, wherein each of the plurality of digits includes a motor having one or more connectors configured to make a contact with the electrical interface when a digit is accommodated in a respective slot.

### TECHNICAL FIELD

The present disclosure relates generally to medical devices; and more specifically, to modular prosthetic devices.

### BACKGROUND

Amputation of a limb, or absence of a limb leads to significant disability for a person to perform routine tasks. This is often addressed by replacing the missing limb with a prosthetic device. Despite recent advancements in the field of prosthetic devices, most commercially available prosthetic limbs leaves much to be desired for an average user. Artificial and prosthetic limbs are used by robots and humans alike as end devices to interact with their environment by implementing movements such as touching, holding, gripping, placing, and the like, with respect to surfaces and/or objects.

While prosthetic devices are a step forward in restoring limb functions, they do come with certain limitations. In a prosthetic device, some elements (such as fingers in case of a prosthetic hand) bear the brunt of most daily wear and tear and are required to be repaired or replaced frequently. However, current prosthetic devices take a long time to repair. Average time to repair may range from 2 to 4 weeks. Moreover, the prosthetic devices often have to be shipped back to the manufacturer/distributor for repair. Meanwhile, a user of the prosthetic device struggles in doing their day to day activities. Furthermore, in case of damage or wear and tear of a particular element of the prosthetic device, often the whole prosthetic device is required to be opened at a service centre for repairs. This adds on to the problem the user faces as they have to wait a long period of time in order to get their prosthetic devices repaired. This time-intensive repair procedure, when required to be performed frequently, leads to considerable inconvenience and expense for the user.

Therefore, in light of the foregoing discussion, there exists a need to overcome the aforementioned drawbacks associated with the existing prosthetic devices.

### SUMMARY

The present disclosure seeks to provide a prosthetic device. An aim of the present disclosure is to provide a solution that overcomes at least partially the problems encountered in prior art.

In one aspect, the present disclosure provides a prosthetic device comprising:
a unitary main body having a plurality of slots, wherein each of the plurality of slots comprises an electrical interface; and
a plurality of digits configured to be detachably connected to the main body, wherein at least one of the plurality of digits comprises a motor having one or more connectors configured to make a contact with the electrical interface when the at least one digit is accommodated in a respective slot.

Embodiments of the present disclosure substantially eliminate or at least partially address the aforementioned problems in the prior art, and provide a modular prosthetic device which enables easy repair and easy replacement of damaged elements therein.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIGS. 1A and 1B are top views of an exemplary prosthetic device, in accordance with an embodiment of the present disclosure;

FIGS. 2A and 2B are schematic illustrations of a prosthetic device, in accordance with different embodiments of the present disclosure; and

FIG. 3 is a schematic illustration of a digit of a prosthetic device, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a prosthetic device comprising:
a unitary main body having a plurality of slots, wherein each of the plurality of slots comprises an electrical interface; and
a plurality of digits configured to be detachably connected to the main body, wherein at least one of the plurality of digits comprises a motor having one or more connectors configured to make a contact with the electrical interface when the at least one digit is accommodated in a respective slot.

The present disclosure aims to provide the prosthetic device. Notably, each of the plurality of digits can be independently and simultaneously attached to or detached from the unitary main body and can be repaired or replaced quickly and without difficulty. In other words, the prosthetic device is modular. In the event of an occurrence of any errors or malfunctions, only the defective digit or the defective components can be replaced, and therefore a technical effect is achieved that the number of parts to be repaired and the time required for a repair are reduced. Moreover, the modular arrangement of components of the prosthetic device increases operational availability of the prosthetic device (namely, prosthesis) as the damaged or worn-out digits can be easily and quickly exchanged in a service centre. Furthermore, the modular nature of the prosthesis is advantageous in increasing the operational availability of the prosthesis to amputees, since malfunctioning modules can readily be identified, isolated, and exchanged/replaced. Consequently, improvements or modifications which are made to the plurality of digits of the prosthetic device from time to time can be easily integrated by a simple exchange of the appropriate module.

The prosthetic device comprises the unitary main body having the plurality of slots, wherein each of the plurality of slots comprises an electrical interface. Herein, the term “unitary main body” refers to a main frame or chassis onto which other elements (such as the plurality of digits) of the prosthetic device are to be attached. Notably, the unitary main body houses several components and acts as a basic structural unit of the prosthetic device. The unitary main body is shaped to emulate a part of a human body. As an example, the unitary main body could be similar to resembling a palm of a human hand. The human hand typically includes an arm portion, a palm portion, and fingers. The palm portion includes a metacarpus (which typically is a group of five bones of the hand between the wrist and the fingers). The metacarpus, in human hands, are flat at a back of the hand and bowed on a palmar side of the hand. For sake of simplicity, the “unitary main body” is also referred to as the “main body”.

Herein, “slots” refer to openings (i.e., empty socket like structures) in the unitary main body into which the plurality of digits can be inserted for attachment. Herein, the process of attaching the plurality of digits (i.e., accommodating the plurality of digits into the plurality of slots) could be done by simply sliding the plurality of digits into their respective slots, rotating (like a screw mechanism) the plurality of digits into their respective slots, using strong magnets (possibly electromagnets), and the like. A number of the plurality of slots that are present in the unitary main body may depend upon the type of prosthetic device being used. Optionally, the number of the plurality of slots is equal to a number of the plurality of digits. For example, when the prosthetic device is a prosthetic hand, the number of the plurality of slots (and the number of the plurality of digits) may be equal to five.

Optionally, a shape of a given slot corresponds to a shape of a given digit that is to be accommodated in the given slot, so as to properly accommodate the given digit therein. Alternatively, optionally, the at least one of the digits comprises a water seal or a jacket configured to at least partially cover a given digit to provide a snug fit between the given digit and a given slot in which the given digit is accommodated. Herein, the water seal or the jacket ensure proper movement of the given digit and a tight fitting with the given slot such that it is not necessary for the given digit to be exactly of the same shape as the given slot. Moreover, the water seal or the jacket further act as a protective layer to the given digit.

Herein, “electrical interface” refers to an element which provides an electrical connection between the unitary main body and at least one of the plurality of digits that are to be attached to the unitary main body. The electrical interfaces are located inside the plurality of slots, and each slot includes one electrical interface. Moreover, the electrical interfaces serve as a linkage flow of electricity between the unitary main body and the at least one of the plurality of digits. A connection between the unitary main body and the at least one of the plurality of digits is established by the electrical interface as and when the plurality of digits are attached to the unitary man body of the prosthetic device.

Preferably, the electrical interface is configured to be connected to a control unit contained in the unitary main body. Notably, the unitary main body houses the control unit. Herein, the “control unit” refers to a specialized unit which is configured to read input from an amputee (i.e., a user of the prosthetic device), process the input, and consequently control the prosthetic device based on the input. The control unit may include, but is not limited to, a receiver for receiving command signals (such as electromyographic (EMG) signals, voice signals, electrical signals, and the like) from the amputee, a processor for processing the command signals to generate corresponding drive signals for controlling the motor, and a memory to store other command related information. Moreover, the purpose of the control unit is to provide the required drive signals for controlling the prosthetic device. Notably, the control unit generates a drive signal corresponding to a received command signal and enables performing of an intended task using the prosthetic device. Additionally, the control unit enables performing of the intended task with a force intended by the user using the prosthetic device. Notably, the electrical interface is connected to the at least one of the plurality of digits and is also connected to the control unit in the unitary main body.

The prosthetic device comprises the plurality of digits configured to be detachably connected to the main body, wherein at least one of the plurality of digits comprises a motor having one or more connectors configured to make a contact with the electrical interface when at least one of the digit is accommodated in a respective slot. Herein, “digits” refer to elements that can be removably attached with the unitary main body of the prosthetic device. The plurality of digits serve as moving parts of the prosthetic device. Furthermore, the number of digits, a type of digit and a scalable size of each digit is fully customizable for the prosthetic device. Additionally, the at least one of the plurality of digits are accommodated in the plurality of slots present in the unitary main body. As an example, the digits may be fingers and thumbs in case of the prosthetic device being implemented as a prosthetic hand. As an example, in case of the prosthetic hand, the unitary body may be implemented as an arm portion and a palm, the palm being provided with five slots. In such a scenario, the five slots acts as sockets (i.e., recesses) for receiving five digits used to implement the fingers and the thumb. Notably, the digits are detachable from the unitary main body. Moreover, technical effect of the digits being detachably connected to the unitary main body is that the plurality of digits can be replaced or repaired quickly and easily by conveniently detaching them from the unitary main body. This is so because the plurality of digits, being moving parts of the prosthetic device, are prone to high wear and tear. Additionally, replacing or repairing a particular digit is much less complicated as compared to opening the whole prosthetic device.

Notably, at least one of the plurality of digits houses a motor. The motor is optionally arranged at a rear end of a given digit, and when the given digit is accommodated in its respective slot, the motor gets accommodated in said slot. Additionally, the motor is equipped with one or more connectors. The one or more connectors are made of a conducting material such that they make a connection with the electrical interface of the unitary main body. The connection is established when the plurality of digits are accommodated in (for example, by sliding said digits into) the plurality of slots. Notably, the motors are responsible for the movement of the digits. The digits are operated by the motor to affect a closing movement, an opening movement, a gripping movement, a tilting movement, and the like, of the digits. Notably, the motors are individually operated in each of the plurality of digits. As an example, one of the digits may close on an object to be grasped before another digit.

Optionally, the prosthetic device comprises a removable locking mechanism to securely hold and remove the digits in their respective slots. Notably, when the prosthetic device is in use, the plurality of digits are accommodated in the plurality of slots in the unitary main body and are held securely by removable locking mechanism. Herein, the removable locking mechanism could be, but is not limited to, a screw locking mechanism, a long pin locking mechanism, and a clasp mechanism. Notably, other varied locking solutions may be used such that the plurality of digits are securely locked into the plurality of slots and are easily removable as and when required. It will be appreciated that such locking mechanisms allow secure attachment as well as quick release of the plurality of digits.

Optionally, the electrical interface comprises at least two contact points to engage with the connectors on the motor. Optionally, in this regard, the at least two contact points are arranged to be physically spaced apart from each other. Notably, inside of the plurality of slots are the electrical interfaces with corresponding at least two contact points for the one or more connectors. Additionally, the at least two contact points act as a conduit for facilitating the electrical connections with the motors in the plurality of digits. The at least two contact points connect with the one or more connectors when the plurality of digits are accommodated in the plurality of slots. Consequently, an electrical connection is established. The at least two contact points are made of an electrically conductive material, and have a shape and size that is suitable according to a shape and size of the plurality of slots.

Optionally, the electrical interface further comprises a motor driver for controlling the motor. Herein, the “motor driver” is a specialized element that controls the operation of the motor. In other words, the motor driver serves as steering electronics associated with the motor. The motor driver may perform functions such as, but not limited to, switching on the motor, switching off the motor, changing a direction of rotation of the motor, changing a speed of the motor, and signal power conversion. When the prosthetic device comprises the control unit, the motor driver serves as an interface between the control unit and the motor. In an example, the motors may require high amount of current to facilitate movement of the plurality of the digits whereas the control unit may work on low current signals. Consequently, the function of the motor driver may be to take a low-current control signal and then turn it into a higher-current signal that can drive the motor. Moreover, the motor driver makes the motor move as per the instructions or inputs given by the control unit. In some implementations, the electrical interface is connected with the motor driver (in a circuit board), whereas in other implementations, the electrical interface is directly connected with the (main) control unit.

Optionally, the connectors on the motor are a plurality of pogo pins or metal springs. Notably, pogo pins and metal springs are a type of electrical connector used to establish electrical connections. As an example, these connectors may be made of brass or copper as a base material on which a thin layer of nickel is applied. As common in electrical connectors, manufacturers often apply a gold plating that improves the durability and contact resistance. The metal springs may be made of copper alloys or spring steel. The plurality of pogo pins or metal springs on the motor are used to establish a reliable electrical connection with the electrical interface.

Optionally, the connectors are disposed on the periphery of the motor. Notably, the connectors are located at the external edge of the motor or external surface of the motor. Additionally, such an arrangement of the connectors helps in establishing connections with the at least two contact points without any intervention. Notably, the location of the at least two contact points in the plurality of slots are such that the connectors come in contact with them as soon as the plurality of digits are accommodated in said slots.

Optionally, the connectors are disposed at a rear-end of the motor. The rear-end of the motor optionally comes into contact with the at least two contact points of the electrical interface, when a given digit is accommodated in its respective slot. Such an arrangement of the connectors helps in establishing connections with the at least two contact points without any intervention.

Optionally, the at least one of the plurality of digits further comprises a worm-gear transmission or a linear mechanism linked to the motor. In this regard, the worm-gear transmission or the linear mechanism is used to provide a controlled application of power, for moving the at least one of the plurality of digits. Herein, the worm-gear transmission comprises a shaft (worm) with a spiral thread that engages with and drives a toothed wheel (worm wheel). Additionally, the worm-gear changes the rotational movement by 90 degrees, and the plane of movement also changes due to the position of the worm on the worm wheel. Notably, the motor applies rotational power to the worm. Consequently, the worm rotates against the worm wheel. Furthermore, the worm wheel moves the digits in desired direction. Herein, the linear mechanism linked to the motor has its stator and rotor unrolled, thus, instead of producing a torque (rotation), it produces a linear force along its length. Notably, the configuration of the worm-gear mechanism or the linear mechanism and the motors in the plurality of digits may provide a drive mechanism that is compact, is capable of providing high torque and has a high precision of movement.

Optionally, the prosthetic device further comprises a four-bar linkage mechanism to operate the at least one digits when powered by the motor via the worm-gear transmission or the linear mechanism. Herein, “four-bar linkage mechanism” is a planar mechanism consisting of four rigid members (i.e., four bodies), that are connected in a loop by four joints, thereby forming a closed-loop kinematic chain of 1-degree-of-freedom. Notably, the four-bar linkage is a movable closed-chain linkage which is applied to the transmission mechanism of each of the plurality of digits. The four-bar linkage mechanism is powered by the motor though the worm-gear transmission or the linear mechanism, and is used to drive the digits. A design of the four-bar linkage mechanism is selected to be such that it guides a wide variety of movements of the at least one of the plurality of digits.

It will be appreciated that the “prosthetic device” refers to a device that replaces a missing body part of a living being. The prosthetic device could be a prosthetic arm, a prosthetic leg, or similar. The prosthetic device may also be used in robotics applications or other powered applications as well because of its human hand-like tactile similarities.

### DETAILED DESCRIPTION OF THE DRAWINGS

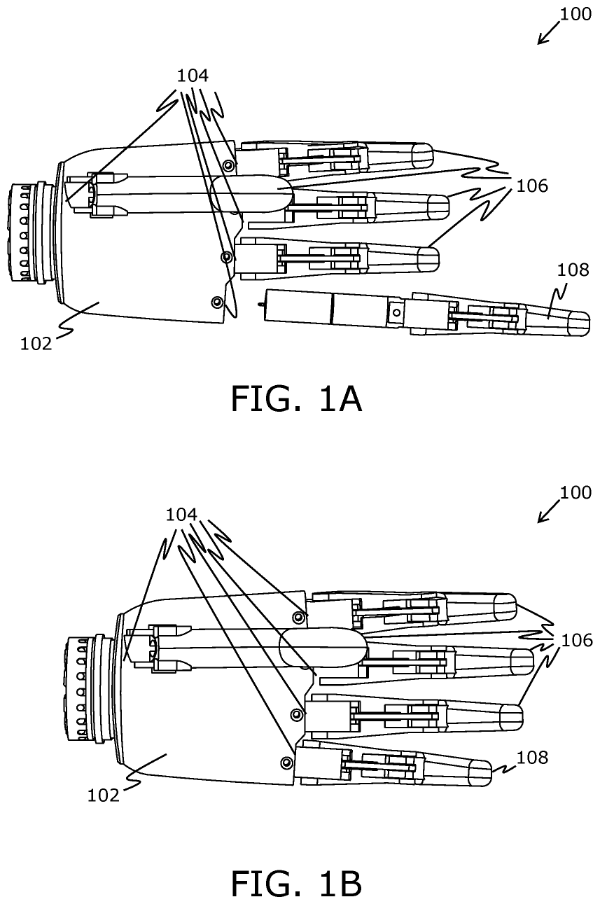

Referring to FIGS. 1A and 1B, illustrated are top views of an exemplary prosthetic device 100, in accordance with an embodiment of the present disclosure. The prosthetic device 100 is shown as a prosthetic hand comprising a unitary main body 102 (depicted for example as a palm portion) having a plurality of slots (depicted as five slots 104), and a plurality of digits (depicted as four digits 106 and one digit 108) configured to be detachably connected to the unitary main body 102. With reference to FIG. 1B, there is shown the one digit 108 that is detached from the unitary main body 102. With reference to FIG. 1C, the one digit 108 is accommodated in its respected slot and is connected to the unitary main body 102.

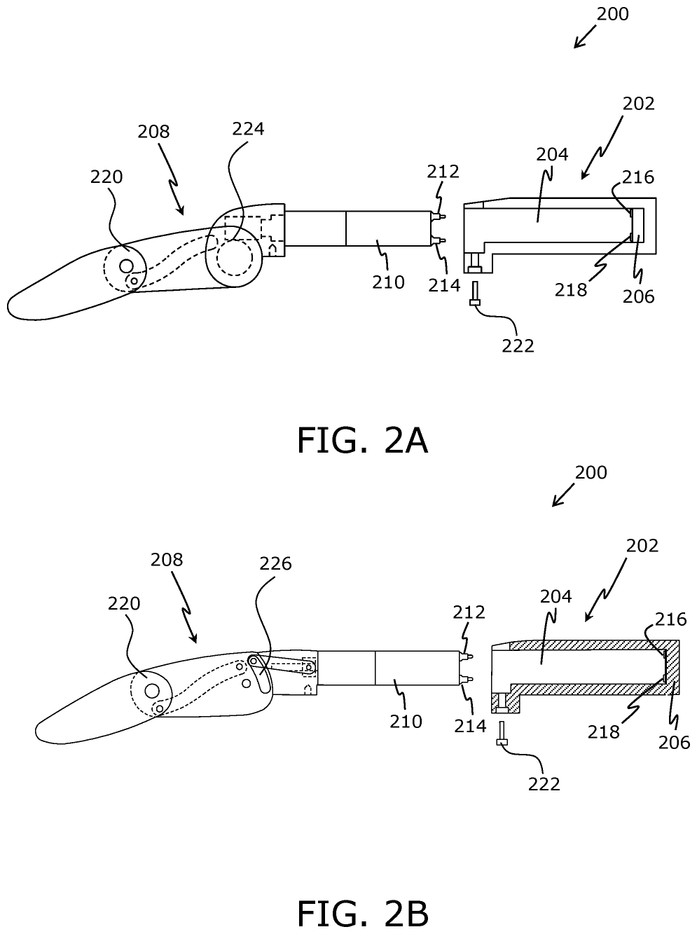

Referring to FIGS. 2A and 2B, illustrated are schematic illustrations of a prosthetic device 200, in accordance with different embodiments of the present disclosure. The prosthetic device 200 comprises a unitary main body 202 having a plurality of slots, such as a slot 204, wherein each of the plurality of slots comprises an electrical interface 206; and a plurality of digits, such as a digit 208 configured to be detachably connected to the unitary main body 202. Each of the plurality of digits comprises a motor 210 having one or more connectors, such as connectors 212 and 214 configured to make a contact with the electrical interface 206 when a digit (such as the digit 208) is accommodated in a respective slot (such as the slot 204).

The prosthetic device 200 is also shown to include that the electrical interface 206 comprises at least two contact points (depicted as contact points 216 and 218) to engage with the connectors 212 and 214 on the motor 210. The connectors 212 and 214 are shown to be disposed at a rear-end of the motor 210. The prosthetic device 200 further comprises a four-bar linkage mechanism 220 to operate the digit 208 when powered by the motor 210 via the worm-gear transmission 220 or the linear mechanism. The prosthetic device 200 further comprises a removable locking mechanism 222 to securely hold and remove the digits (such as the digit 208) in their respective slots (such as the slot 204). In FIG. 2A, the digit 208 is shown to further comprise a worm gear transmission 224 linked to the motor 210. In FIG. 2B, the digit 208 is shown to comprise a linear mechanism 226 linked to the motor 210.

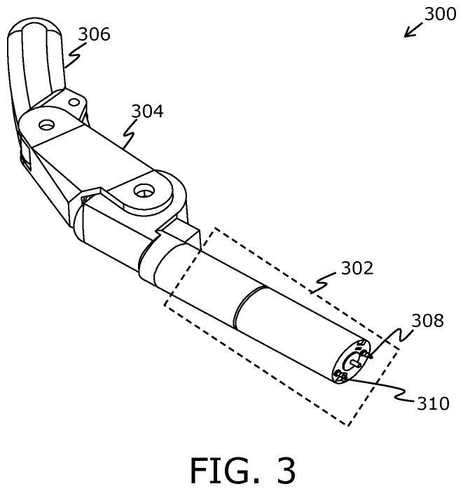

Referring to FIG. 3, illustrated is a schematic illustration of a digit 300 of a prosthetic device (not shown), in accordance with an embodiment of the present disclosure. When the digit 300 is accommodated in a corresponding slot in a unitary main body (not shown) of the prosthetic device, a portion 302 of the digit 300 is accommodated in the corresponding slot whereas a remaining portion of the digit 300 extends out of the unitary main body. Notably, the portion 302 of the digit 300 houses a motor (not shown). The motor is arranged at a rear end of the digit 300, and when the digit 300 is accommodated in the corresponding slot, the motor gets accommodated in said slot. Additionally, the digit 300 further comprises a worm-gear transmission (not shown) or a linear mechanism (not shown) linked to the motor. The worm-gear transmission or the linear mechanism is used to provide a controlled application of power, for moving the digit 300. Herein, the worm-gear transmission comprises a shaft (worm) with a spiral thread that engages with and drives a toothed wheel (worm wheel). In an exemplary implementation, the shaft (worm) is housed in the portion 302 of the digit 300 and the toothed wheel (worm wheel) is housed, in a sub-portion 304 of the remaining portion of the digit 300. Furthermore, the digit 300 comprises a four-bar linkage mechanism (not shown) to operate the digit 300 when powered by the motor via the worm-gear transmission or the linear mechanism. The four-bar linkage mechanism is housed, for example, in a sub-portion 306 of the remaining portion of the digit 300, and enables various movements of the digit 300. The digit 300 is also shown to include connectors 308 and 310 to engage with at least two contact points (not shown). The connectors 308 and 310 are shown to be disposed at a rear-end of the digit 300.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A prosthetic device comprising:
a unitary main body having a plurality of slots, wherein each of the plurality of slots comprises an electrical interface; and
a plurality of digits configured to be detachably connected to the main body, wherein at least one of the plurality of digits comprises a motor having one or more connectors configured to make a contact with the electrical interface when the at least one digit is accommodated in a respective slot.

2. The prosthetic device of claim 1, further comprises a removable locking mechanism to securely hold and remove the digits in their respective slots.

3. The prosthetic device of claim 1, wherein the electrical interface comprises at least two contact points to engage with the connectors on the motor.

4. The prosthetic device of claim 3, wherein the electrical interface further comprises a motor driver for controlling the motor.

5. The prosthetic device of claim 1, wherein the electrical interface is configured to be connected to a control unit contained in the main body.

6. The prosthetic device of claim 1, wherein the connectors on the motor are a plurality of pogo pins or metal springs.

7. The prosthetic device of claim 6, wherein the connectors are disposed on the periphery of the motor.

8. The prosthetic device of claim 7, wherein the connectors are disposed at a rear-end of the motor.

9. The prosthetic device of claim 1, wherein the at least one of the plurality of digits further comprises a worm-gear transmission or a linear mechanism linked to the motor.

10. The prosthetic device of claim 9, further comprises a four-bar linkage mechanism to operate the at least one digit when powered by the motor via the worm-gear transmission or the linear mechanism.

11. The prosthetic device of claim 1, wherein the at least one of the digits comprises a water seal or a jacket configured to at least partially cover a given digit to provide a snug fit between the given digit and a given slot in which the given digit is accommodated.
